(19) United States
(12) Patent Application Publication
(10) Pub. No.: US 2023/0090686 A1
(43) Pub. Date: Mar. 23, 2023

(54) PHOTOBIOMODULATION SYSTEM AND METHOD FOR IMPROVED IMMUNITY AND TREATMENT OF RESPIRATORY TRACT INFECTIONS

(71) Applicant: Lew Lim, Toronto (CA)

(72) Inventor: Lew Lim, Toronto (CA)

(21) Appl. No.: 17/798,405

(22) PCT Filed: Mar. 22, 2021
(86) PCT No.: PCT/IB2021/052349

§ 371 Date: Aug. 9, 2022

Related U.S. Application Data
(60) Provisional application No. 63/004,752, filed on Apr. 3, 2020.

(51) Int. Cl. A61N 5/06

(52) U.S. Cl. CPC A61N 5/0624; A61N 5/0603; A61N 2005/0626; A61N 2005/0664; A61N 2005/0607; A61N 2005/0659; A61N 2005/0663

(57) ABSTRACT

A self-administrable system for improved immunity and treatment of respiratory tract infections in a subject, said system comprising: a configured irradiation unit for delivery of light energy into at least one portion of an in-vivo target selected from the group consisting of the thymus gland, sternal bone marrow and lungs.

### FIELD OF THE INVENTION

The present invention relates to photobiomodulation, and more specifically, to a photobiomodulation system and method for treatment of respiratory tract infections.

### BACKGROUND ART

Respiratory Tract Infections

A respiratory tract infection (RTI) is an infectious disease involving the respiratory tract. An infection of this type can be classified as an upper respiratory tract infection (URI) or a lower respiratory tract infection (LRI).

The upper respiratory tract is generally considered to be the airway above the glottis or vocal cords. This part of the tract includes the nose, sinuses, pharynx and larynx. Typical infections of the upper respiratory tract include tonsillitis, pharyngitis, laryngitis, sinusitis, otitis media, certain types of influenza, and the common cold. Symptoms of URIs can include cough, sore throat, runny nose, nasal congestion, headache, low grade fever, facial pressure and sneezing.

The lower respiratory tract consists of the trachea (wind pipe), bronchial tubes, the bronchioles and the lungs. Lower respiratory tract infections are generally more serious than upper respiratory infections. LRIs are the leading cause of death among all infectious diseases. The two most common LRIs are bronchitis and pneumonia.

COVID-19

COVID-19 is the infamous respiratory tract infection responsible for a worldwide pandemic beginning in 2019. As of early 2021, several vaccines with varying efficacies had been developed. However, there have been many problems with rolling out the vaccines. Many countries, including high-income countries, are dealing with vaccine shortages even to vaccinate the highest risk and vulnerable members of their populations.

Furthermore, because they have a high propensity to mutate, many versions of a virus can exist. This makes viruses perpetual moving targets for synthetic treatment intervention. For example, as a result of mutations, the main genotype in this pandemic, SARS-CoV-2, differs substantially from other viruses such as those that cause the common cold including four other types of coronaviruses (OC43, HKU1, NL63, and 229E) and the various influenza variants. This is why treatments effective against well known viruses are often not effective against novel viruses such as SARS-CoV-2. The same issue arises for SARS-CoV-2 as new variants emerge.

Even as vaccines against COVID-19 have been developed, variants have emerged at the same time. This includes variants from the United Kingdom and South Africa which some experts fear to be more easily transmissible and could possibly cause a higher mortality rate. It is uncertain whether the current vaccines are as effective against these new variants. There is therefore a case for an intervention that is potentially agnostic to variants of coronaviruses.

Photobiomodulation (PBM)

Photobiomodulation (PBM), also known as low-level light therapy (LLLT), is a biostimulation technique that delivers photons (mainly of red and near infrared wavelengths) to living tissues to modulate their functions. It may even have promise in strengthening the immune system. The growth factors expressed in PBM activity accelerates the healing of tissues.

The biochemical mechanisms of PBM interaction include increasing the activity of ion channels such as the Na+/K+ ATPase and the indirect effects include regulating important secondary messengers such as calcium, cyclic adenosine monophosphate (cAMP) and reactive oxygen species (ROS)—all of which result in diverse biological cascades. These biological cascades lead to effects such as the maintenance of homeostasis and activating protective, anti-oxidant and proliferative gene factors, as well as the systematic responses, such as cerebral blood flow, which is deficient in neurocognitive disorders.

The most well investigated mechanism of action of PBM is its fundamental effect on mitochondrial function. PBM has been demonstrated to increase the activity of complexes in the electron transport chain of mitochondria, comprising complex I, II, III, IV and succinate dehydrogenase. In complex IV, the enzyme cytochrome c oxidase (CCO), functions as photo acceptor as well as transducer. CCO specifically accepts and transduces light in the red (620-700 nm) and the near-infrared (780-1400 nm), wavelengths of lights which can be processed in PBM. The process increases the amount of ATP produced, as well as cyclic adenosine monophosphate (cAMP) and reactive oxygen species (ROS). The increase in ATP increases the activity of ion channels regulating cAMP and calcium, which results in the stimulation of diverse biological cascades and activate up to 110 genes for transcription, which leads to healing and recovery activities and the prolongation of the production of energy by the mitochondria. One of the most prominent responses to PBM is the activation of sodium pumps and the Na+/K+ ATPase, which leads to greater membrane stability and resistance to depolarization.

It would be advantageous to use a non-invasive therapy, such as PBM, for the treatment of respiratory tract infections, such as COVID-19.

### SUMMARY OF THE INVENTION

In one aspect, the present invention provides a system for improved immunity and treatment of respiratory tract infections in a subject, said system comprising:

a configured irradiation unit comprising a portable hollow casing having fixed dimensions, a sized internal spatial volume and an external surface configuration suitable for application to the chest, said portable hollow casing of the configured irradiation unit being comprised of:

(i) a light energy transmitting material which forms at least a portion of the configured external surface for said hollow casing of the configured irradiation unit; and (ii) at least one light generating unit housed and contained within said internal spatial volume of said hollow casing of the configured irradiation unit and which is capable of generating light energy of at least one preselected wavelength selected from the group consisting of near infrared light wavelengths and visible red light wavelengths, at a predetermined energy intensity, for a preset time duration, and at a predetermined pulse frequency, collectively on-demand sufficient to penetrate through the skin and to pass to at least one portion of an in-vivo target selected from the group consisting of a thymus gland, sternal bone marrow and lungs,

whereby said configured irradiation unit can emit light energy after application to the chest and achieve passage of said emitted light energy through the skin into the at least one portion of the in-vivo target; a frame adapted for support of said configured irradiation unit and for at will placement of said light transmitting external surface of said configured irradiation unit at a fixed position and desired irradiation direction on the chest;

a portable controller assembly able to control on-demand delivery of light energy from said configured irradiation unit into at least one portion of the thymus gland, sternal bone marrow and/or lungs in-vivo, said controller assembly including:

(a) a power source of on-demand direct electrical current,
(b) a central processing unit for controlling and directing the flow of such direct electrical current,
(c) at least one connector in electrical communication with the power source for on-demand conveyance of direct electrical current to the central processing unit, and
(d) at least one connector in electrical communication with the configured irradiation units for on-demand conveyance of direct electrical current from said central processing unit to said light generating units.

In another aspect, the present invention provides a method for improved immunity and treatment of respiratory tract infections in a subject, said method comprising the steps of:

A. obtaining a light energy-emitting apparatus comprised of:

a configured irradiation unit comprising a portable hollow casing having fixed dimensions, a sized internal spatial volume and an external surface configuration suitable for application to the chest, said portable hollow casing of the configured irradiation unit being comprised of:

(i) a light energy transmitting material which forms at least a portion of the configured external surface for said hollow casing of the configured irradiation unit; and (ii) at least one light generating unit housed and contained within said internal spatial volume of said hollow casing of the configured irradiation unit and which is capable of generating light energy of at least one preselected wavelength selected from the group consisting of near infrared light wavelengths and visible red light wavelengths, at a predetermined energy intensity, for a preset time duration, and at a predetermined pulse frequency, collectively on-demand sufficient to penetrate through the skin and to pass to at least one portion of an in-vivo target selected from the group consisting of the thymus gland, sternal bone marrow and lungs,

whereby said configured irradiation unit can emit light energy after application to the chest and achieve passage of said emitted light energy through the skin into the at least one portion of the in-vivo target;

a frame adapted for support of said configured irradiation unit and for at will placement of said light transmitting external surface of said configured irradiation unit at a fixed position and desired irradiation direction on the chest;

a portable controller assembly able to control on-demand delivery of light energy from said configured irradiation unit into at least one portion of the thymus gland, sternal bone marrow and/or lungs in-vivo, said controller assembly including:

(a) a power source of on-demand direct electrical current,
(b) a central processing unit for controlling and directing the flow of such direct electrical current,
(c) at least one connector in electrical communication with the power source for on-demand conveyance of direct electrical current to the central processing unit, and
(d) at least one connector in electrical communication with the configured irradiation units for on-demand conveyance of direct electrical current from said central processing unit to said light generating units; and
B. causing said light generating units of said positioned configured irradiation unit to generate light energy of at least one preselected wavelength selected from the group consisting of near infrared light wavelengths and visible red light wavelengths, at a predetermined energy intensity, for a preset time duration, and at a predetermined pulse frequency, collectively on-demand sufficient to penetrate through the subject's skin and to pass into the at least one portion of the in-vivo target.

### DESCRIPTION OF THE DRAWINGS

The present invention may be better understood and more readily appreciated when taken in conjunction with the accompanying drawings, in which:

FIG. 1 is a perspective view of a preferred system of the present invention;

FIG. 2 is another perspective view of a preferred system of the present invention;

FIG. 3 is a side view of a preferred system of the present invention applied to a subject; and

FIG. 4 is a front view of a preferred system of the present invention applied to a subject.

### DETAILED DESCRIPTION OF THE INVENTION AND PREFERRED EMBODIMENTS

Use of PBM Against Viral Infections

The human body has repeatedly shown to have the ability to adapt to ever-morphing microbes and viruses. The ability to overcome these moving targets rests heavily on the state of the immune system. It is therefore sensible to invest in a way to support and enhance the natural intelligence of the body's immune system. The inventor recognizes that a PBM modality can be one such solution because it supports the body's natural intelligence to restore functional homeostasis, and at the same time, enhances the immune response to infections such as COVID-19.

The inventor proposes that PBM can treat viral infections by strengthening the immune system. Further, PBM reduces inflammation and the overactivation of inflammatory cytokines that are characteristic of COVID-19 cases. The growth factors expressed in PBM activity accelerates the healing of tissues that are damaged in severe infections and inflammatory responses.

Components of the Preferred System/Apparatus

The system and apparatus of the present invention preferably comprises at least the following component parts:

(1) a portable hollow casing;
(2) one or more light generating units which are housed and contained within the interior spatial volume of the hollow casing;
(3) a source of electrical current;
(4) a process controller assembly; and
(5) optionally, a smart phone, tablet computer or other computing device.

These components may preferably be electrically linked together by at least one connector for transfer of direct electrical current from the source of electrical current to the controller assembly, and at least one connector for conveyance of direct electrical current from the controller assembly to the light generating unit.

1. Portable Hollow Casing

The present invention includes at least one portable hollow casing having fixed dimensions, a sized internal spatial volume and an external surface configuration suitable for application to the subject. The intended purposes and goals of the portable casing are twofold: (i) to serve as a containment chamber that is configured for easy application to the subject; and (ii) to act as a molded lens that reflects and directs emitted light waves to the subject.

Preferably, the portable casing may be constructed and formed of a light transmitting material over at least a portion of its external surface, and will encompass that volumetric zone intended for housing and containment of at least one light generating unit. By definition, such light transmitting material includes and encompasses transparent, translucent and opaque matter. However, in most instances, a completely clear and transparent matter is preferred.

2. Light Generating Unit(s)

The light generating unit will be able to deliver therapeutic light at wavelengths that include but are not necessarily limited to the following: (i) in the visible color spectral ranges, the visible red light wavelengths ranging between about 620-780 nm; and (ii) in the non-visible spectral ranges, the near-infrared light wavelengths ranging between about 780-1400 nm. In addition, the generated light energy waves and particles may alternatively be: (i) either coherent (as in lasers) or non-coherent (as in non-laser light emitting diodes (LEDs); (ii) be either pulsing or non-pulsing (continuous wave) in delivery; (iii) be either constant or non-constant in intensity; (iv) be either uniform or non-uniform in phase; (v) polarized and non-polarized; and (vi) have a regular or irregular flux.

Any conventionally known means for generating electromagnetic radiation or articles for propagating radiant energy are acceptable for use in the present apparatus. In the majority of embodiments, it is intended and expected that either a low level laser unit or a LED will be employed as the light generating unit(s) for irradiating purposes.

3. Source of Electric Current

It is preferred that a portable and replenishable source of on-demand direct electrical current exist as a component part of the apparatus and system of the present invention. The therapeutic treatment system and method provided by the instant invention is intended to deliver a specific energy dosage (measured in Joules), which is a function of power (in wattage) and time (in seconds), and which is deemed to be efficacious for each therapeutic treatment.

The power supply typically will convey energy in the form of direct electric current. Adequate quantities of electric current can be repeatedly conveyed from, for example, a single battery source or from a combination of several dry cells joined together in series or parallel. In some other desirable embodiments, the source of electric power will be in the form of a rechargeable power bank, a direct current battery unit (rechargeable from ordinary household alternating current receptacles) or as alternating current (AC) via a power adaptor. It is expected and intended that there will be several alternative embodiments with different combinations of these components and which would be suitable for different configurations of power, energy dosage and treatment time.

As to positioning, in some preferred embodiments, the power source is a discrete entity which is held and contained entirely within the internal confines of the controller assembly. In other preferred embodiments, however, the source of electric current can be a self-contained, separate and free standing unit which is in electrical communication with the controller assembly via an electrical cable and connector module linkage, such as a portable and rechargeable power bank. In an alternative embodiment, the source of electrical current is obtained by plugging the system and apparatus into the local electrical grid via a power adaptor.

4. Process Controller Assembly

The process controller assembly is a portable unit component having at least three structural features:

(i) A receiving circuit for receipt of such electrical current as is transferred to the controller assembly from the electrical current source;
(ii) A central processing unit (CPU) for controlling and directing the flow of such electrical current as is received by the controller assembly over time; and
(iii) A delivery circuit for delivering direct electrical current from the controller assembly to the light generating unit(s).

It is intended and expected that the process controller assembly will be electrically linked to other essential components of the apparatus and thus typically will also have:

(a) at least one connector for transfer of direct electrical current from the source of electrical current to the controller assembly; and
(b) at least one connector for conveyance of direct electrical current from the controller assembly to the light generating unit(s).

These connectors typically are formed as insulated copper wire cables and jack modules that allow for quick and easy linkage and electrical communication with both the electrical current source and the light generating unit(s).

It is intended and expected that any conventionally known and interchangeable electric cables and connectors will be used to link the controller assembly to the irradiation lens. This also provides a distinct advantage and benefit to the user, namely the option to exchange one configured irradiation lens (able to transmit light at a first wavelength) for another irradiation lens (able to transmit light at a second and different wavelength), and thereby permits the use of different lasers and alternative light emitting diodes able to deliver different wavelengths of visible and invisible light energy with one single controller assembly.

In some preferred embodiments, the source of electrical current lies internally and is contained within the interior spatial volume of the controller assembly, and appears as an electric battery (dry cell or rechargeable unit). In this instance, the controller assembly also has a socket adapted for the attachment of an insulated copper wire cable and modular jack connector, whose other end is joined to the light generating unit disposed within the hollow casing.

The central processing unit (“CPU”) of the controller assembly is preferably able to regulate light energy with respect to many different parameters including but not limited to: wavelength, coherency/synchrony, energy (Joules (J)), Power (Watts (W) or milliwatts (mW)) or irradiance (W/cm²), radiant exposure (J/cm²), exposure time (seconds), pulse mode (continuous or pulse), frequency (Hertz (Hz)), duty cycle (percentage), fraction protocol (number of patient treatment sessions), light beam size (area of landed beam), and light beam penetration (delivery) distance.

The process controller assembly will not operate in the absence of a source of electrical current. In addition, the controller assembly, besides preferably switching off the unit after a predetermined time, is a circuitry which provides power to drive the light generating unit(s) properly and efficiently. The controller also ensures that the power delivered to the light generating unit(s) is consistent. It therefore desirably monitors the battery strength where the source is a power bank or battery, and switches off the unit if the power bank or battery is unable to supply sufficient power to drive the circuitry properly.

In a preferred embodiment, the controller is part of the same part of the system which houses the light generating unit(s). Alternatively, the controller is detached from this part but connected via a cable for communication.

5. Smart Phone, Tablet Computer or Other Computing Device

In one alternative embodiment, the function of the controller assembly is controlled, in whole or in part, by smartphone, smartwatch, tablet computer, laptop computer, desktop computer or any appropriate computing device. The smart phone, for example, may operate on one of the more popular mobile platforms. The light generating unit(s) could be connected via a cable or wirelessly to the smart phone. The smart phone carries a downloadable software application that would largely duplicate the software functions in the controller assembly. A modified attachment containing interface processing software in a computer chip will provide a physical connection between the controller and the proprietary smart phone platform. The software application will also contain more software controls and graphic interfaces. Alternatives to the smart phone include a smartwatch, tablet computer, laptop computer, desktop computer or any appropriate computing device with the software application downloaded thereon.

In yet another alternative embodiment, the controller assembly works in combination with smartphone, smartwatch, tablet computer, laptop computer, desktop computer or any appropriate computing device. In particular, the computing device has downloaded thereon a software application which can: (i) turn the controller assembly on and off; and/or (ii) transmit instructions to the controller assembly to adjust the light energy parameters of each individual light generating unit, including but not limited to wavelength, coherency/synchrony, energy (Joules (J)), Power (Watts (W) or milliwatts (mW)) or irradiance (W/cm²), radiant exposure or dose or fluence density (J/cm²), exposure time (seconds), pulse mode (continuous or pulse), frequency (Hertz (Hz)), duty cycle (percentage), fraction protocol (number of patient treatment sessions), light beam size (area of landed beam), and light beam penetration (delivery) distance.

Furthermore, the computing device can serve as a system interface where a user enters instructions through the interface to turn the controller assembly on and off and/or adjust the light energy parameters of each individual light generating unit. Instructions may be entered by any known input component such as a touch screen, mouse, keypad, keyboard, microphone, camera or video camera. Once the user inputs instructions into the system interface, instructions are transmitted to the controller assembly which then adjusts the parameters of the light energy being delivered to by the light generating units.

In these embodiments, any conventionally known and interchangeable electric cables and connectors can be used to link the computing device to the controller assembly. Alternatively, the computing device may communicate with the controller assembly by wireless means. Connections between any of these components are implemented using appropriate wired or wireless communications via protocols such as BLUETOOTH™, Wi-Fi, Near Field Communications (NFC), Radio Frequency Identification (RFID), 3G, Long Term Evolution (LTE), Universal Serial Bus (USB) and other protocols and technologies known to those skilled in the art.

Two specific components of a preferred system of the present invention could have a role in the treatment of RTIs, such as COVID-19:

1. An intranasal LED device applied in the nasal cavity; and
2. A LED module positioned on the sternum.

Both specific components of the preferred system of the present invention may also contribute an additional beneficial systemic effect that is characteristic of PBM.

Mechanism of Action

The system of the present invention delivers light of specific wavelength, power and duration to the body. The body responds by utilizing the energy to translate numerous interacting elements to restore functional homeostatic balance. A beneficial outcome is the regulation of the immune system. PBM using the system of the present invention elevates a weakened immune system, and in cases of healthy individuals, is prophylactic.

The fundamental mechanism of action of PBM is based on directing light photons to the mitochondria at the cellular level. PBM has a modulating action on the mitochondrial respiratory chain where a transient release of non-cytotoxic levels of reactive oxygen species (ROS) leads to positive effects. PBM has a regulatory role via crosstalk with nuclear factor kappa-light-chain-enhancer of activated B cells (NF-κB) for the management of various conditions, including immune-related conditions. In an immune-compromised system, the chain of activity leads to increased production of appropriate levels of white blood cells, while managing inflammation. The appropriate dose of PBM directed to the mitochondria can positively modulate the immune system.

Action Again COVID-19

Regarding COVID-19, the corona spike protein of the SARS-COVID membrane has a higher propensity to absorb light from ultraviolet to infrared. This process can transform the envelope of the virus and thus weaken it. This makes the virus particularly amenable to further action of PBM in the red and NIR spectra used by the system of the present invention.

Another relevant component released during PBM is nitric oxide (NO). It is commonly identified with vasodilatation and improved blood circulation. However, in the context of a coronavirus pandemic, its value to potentially inhibit the replication of coronavirus is much more important.

In view of the effect of PBM on the electron transport chain of mitochondria, such as on the enzyme cytochrome c oxidase (CCO), the inventor proposes an inhibitory effect on coronavirus replication. The function of CCO as a photo acceptor is enhanced when photons from the PBM process dissociates nitric oxide (NO) from the CCO. NO can then inhibit the replication of coronaviruses.

Preferred Targets for System/Apparatus of the Present Invention

### A. Thymus Gland

In PBM, low levels of red and NIR light interact with cells to cause changes at the molecular, cellular and tissue levels. Besides restoration of immune function, PBM leads to stem cell genesis that progress to embryonic cells for tissue repair and production of white blood cells to support the immune system.

The application of a controlled dose of PBM to the thymus gland can cause improved maturation of T-lymphocytes. Although, the thymus gland shrinks as we age, PBM activates the remaining gland and surrounding bone marrow to contribute to mesenchymal stem cell genesis. The overall effect helps to boost the immune system to resist a viral infection.

The system of the present invention has a LED module placed over the thymus gland to stimulate the production of T-lymphocytes and surrounding bone tissues.

B. Upper and Lower Respiratory Tracts

Some viral respiratory infections, such as COVID-19, affects both the upper respiratory tract (nasal cavity, pharynx, larynx) and the lower respiratory tract (trachea, primary bronchi, lungs). One of the reasons for the potency of COVID-19 is its ability to migrate to the lungs and access the host type II alveolar cells, the most abundant type of alveoli where gas exchanges take place. Its entry is facilitated through the enzyme ACE2 connected by its “corona” spikes. As alveolar damage progresses, respiratory failure ensues and death may follow.

The present invention may preferably include direct irradiation of the lower respiratory tract, particularly the lungs where most of the COVID-19 consequential pathology takes place in a symptomatic patient. One outcome from PBM-related mitochondrial action is the release of nitric oxide (NO) dissociated from the respiratory chain. In viral infections, NO effects are complex and can be protective or deleterious. However, in our examination of the COVID-19 pathology, it has been found that NO has the beneficial effect of inhibiting the replication cycle of SARS-CoV.

In the present invention where the lungs are targeted, PBM is preferably directed to regions in the chest, more preferably around the sternum. This is the same as the preferred position of the LED module to target the thymus gland. Therefore, this position allows for the double effect of irradiating the thymus, as well as the lungs. The LED module of the system of the present invention for this area emits light of preferably about 810 nm. This wavelength was chosen for its penetration depth into mammalian tissues with minimum absorption by water.

C. Nasal Cavity

The nasal cavity has been chosen to preferably position a LED because of the dense blood capillary networks shielded by very thin membrane. This makes the area relatively easy for light from the system of the present invention low-power LED to reach the blood circulatory system and the requisite tissue.

PBM has a body-wide systemic effect mediated by omnipresent circulating cell-free respiratory competent mitochondria. This is in addition to the mitochondria embedded inside human eukaryotic cells. Therefore, the positive effect of therapeutic light is delivered throughout the body by simply by lighting up the blood capillaries in the nasal cavity where these circulating mitochondria are present. This effect is circulated and spread throughout the body through the major vessels at about three times a minute.

A study using red laser optic fiber to treat vasomotor rhinitis presented a significant increase of T-lymphocytes. The complex and cascading mechanisms that start from the free-floating circulating mitochondria in the blood vessels surrounding the nasal cavity are the likely factors behind the outcomes. PBM via the nasal cavity using the system of the present invention can boost the production of the protective white blood, including T-lymphocyte cells, which are present throughout the body.

The intranasal applicator of the system of the present invention is used such that its LED preferably delivers red light at 633 nm at a safe power density of 6.5 mW/cm2.

There are suggestions that ultraviolet (UV) C may help to eliminate viruses by direct irradiation but prolonged exposure has carcinogenic risks, so the most preferred wavelength to be used in the nasal cavity should be subject to further investigations.

In summary, the application of PBM to the nasal cavity and thymus gland, with light in the red and NIR range, activates the body's immune response system throughout the whole body. These wavelengths also fall within the range and around the peak of action spectra for PBM effects. For this effect, the system of the present invention has an intranasal applicator to preferably deliver 633 nm wavelength. Its depth of penetration is estimated to be near optimum for penetrating and irradiating the vascular network under the thin membrane around the nasal cavity.

Further Applications of System/Apparatus of Present Invention

Cytokine Storm Syndrome

Accumulating evidence suggests that a subgroup of patients with severe COVID-19 might have cytokine storm syndrome. A “cytokine storm” is an overproduction of immune cells and their activating cytokines, which is often associated with a surge of activated immune cells into the lungs. The resulting lung inflammation and fluid buildup can lead to respiratory distress and can be contaminated by a secondary bacterial pneumonia, often increasing the mortality in patients.

It is vital to manage the lung inflammation and the contributing cytokines. PBM can increase immune activation by promoting NF-κB proteins in normal cells. In the presence of inflammatory markers, PBM can have anti-inflammatory effects. The anti-inflammatory characteristic of PBM is expected to calm a potential cytokine storm in patients.

Sepsis after Infection

Damage to the lungs due to an infection could lead to the subsequent risk of sepsis due to a weakened immune system working on overdrive. Statistics show that half of the survivors of such infection suffer from further infections, kidney failure or cardiovascular problems about three months after the incidence.

Furthermore, many sepsis patients suffer severe, long-term functional, cognitive or psychological consequences such as paralysis, depression or anxiety disorders. In 2017, the global burden of sepsis accounted for 49 million cases and 11 million deaths.

In an animal study, the findings suggested that PBM is an inexpensive and non-invasive treatment for sepsis that could be effective.

Prophylaxis Against Disease

PBM has been shown to modulate the body's own immune response, both locally and systemically. The fact that PBM strengthens the immune system makes it a credible prophylaxis against diseases, which include viral infections.

Preferred Embodiment of the System of the Present Invention

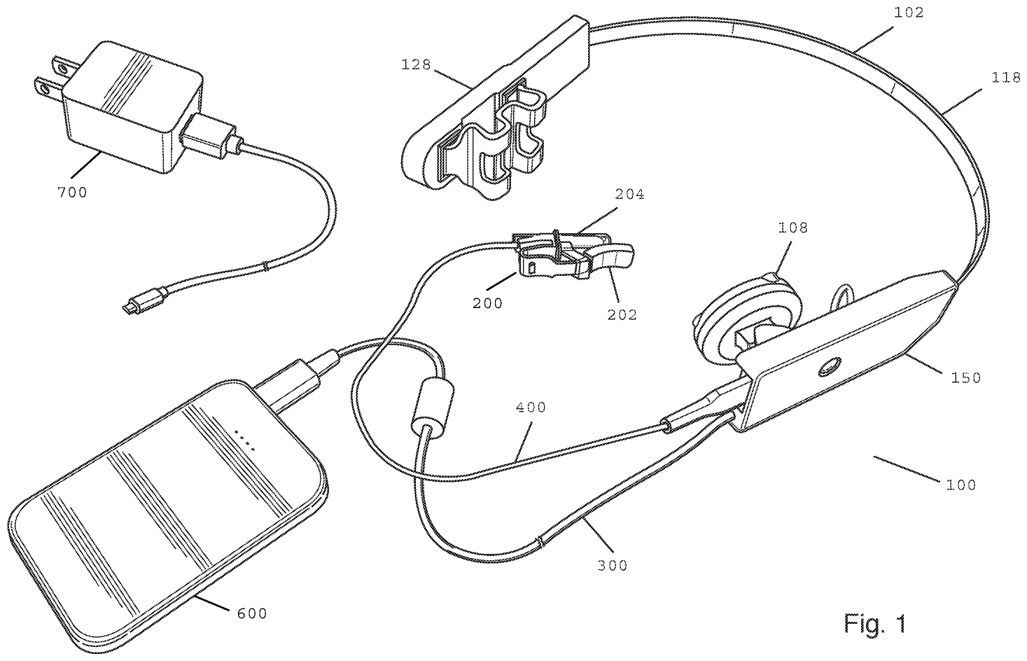

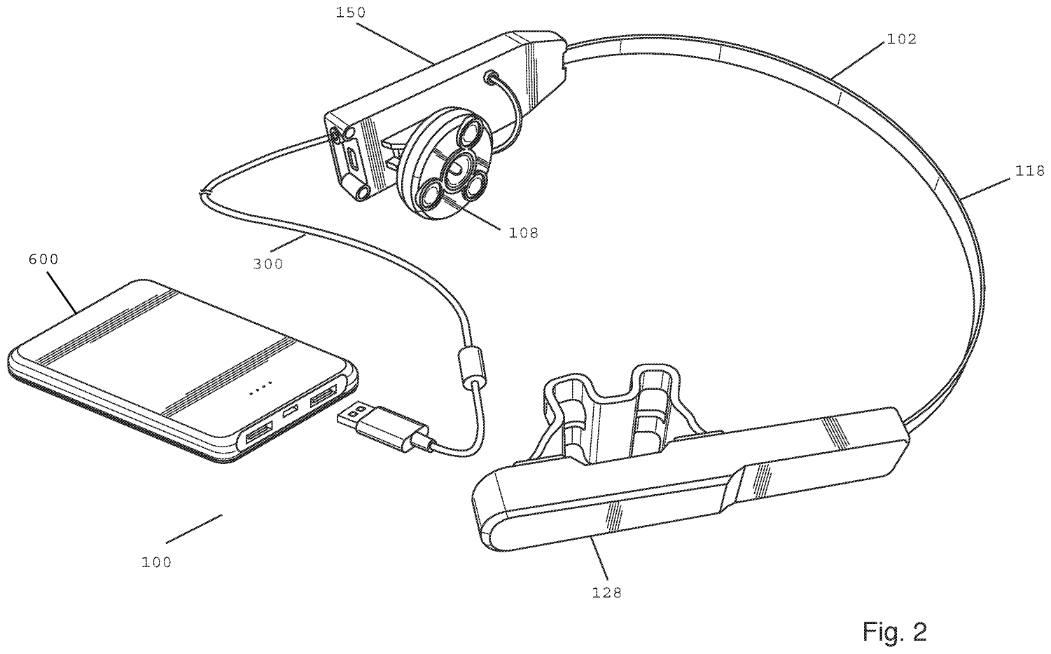

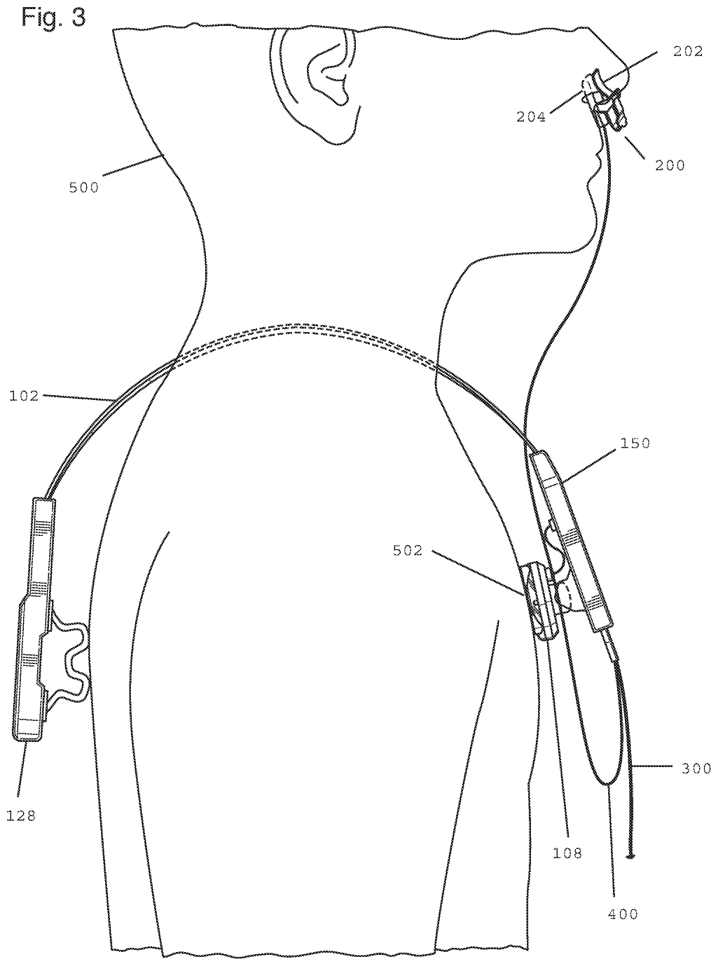

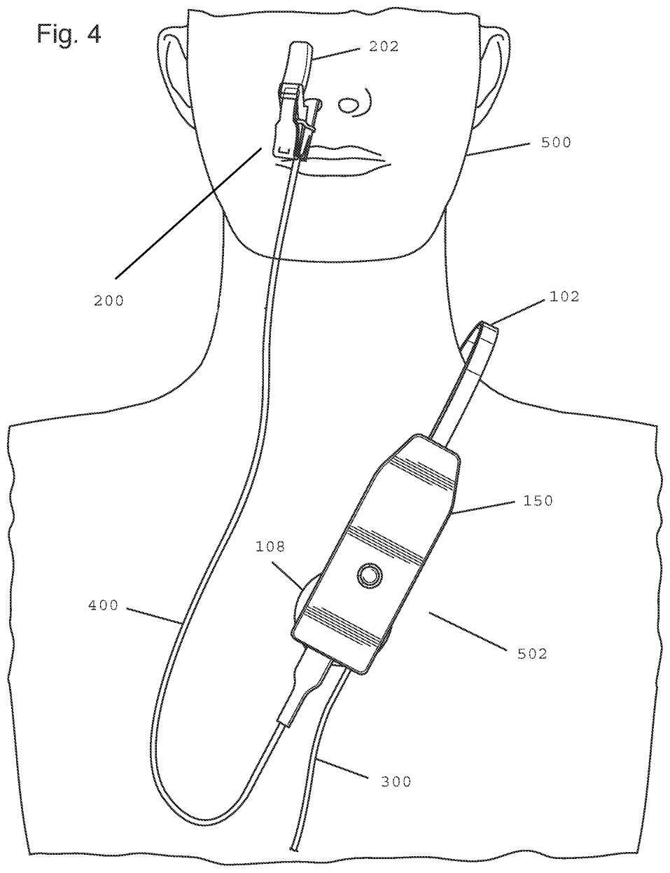

As shown in FIGS. 1 to 4, the present invention provides a preferred embodiment of an apparatus 100 having a respiratory tract light therapy unit 102. Optionally, as can be seen in FIGS. 1 and 3 to 4, there is an intranasal unit 200 as well.

A controller assembly 150 can serve as a power source and central processing unit for both the respiratory tract light therapy unit 102 and intranasal unit 200. In the preferred embodiment shown in FIGS. 1 to 4, the controller assembly 150 is located on the respiratory tract light therapy unit 102. In alternative embodiments, the controller assembly 150 is a separate unit which can communicate with the respiratory tract light therapy unit 102 and intranasal unit 200.

Referring to FIGS. 1 to 4, the respiratory tract light therapy unit 102 comprises one or more configured irradiation units 108, each of the configured irradiation unit 108 including a portable hollow casing having fixed dimensions, a sized internal spatial volume, and an external surface configuration suitable for application to the chest 502 of the subject 500.

The portable casing comprises: (i) a light energy transmitting material which forms at least a portion of the configured external surface for said hollow casing, and (ii) at least one light generating unit entirely housed and contained within said internal spatial volume of said hollow casing and which is capable of generating light energy of at least one preselected wavelength selected from the group consisting of near infrared red light wavelengths and visible red light wavelengths, at a predetermined energy intensity, for a preset time duration, and at a predetermined pulse frequency, collectively on-demand sufficient to penetrate through the chest 502 and to pass into the body.

A frame 118 is provided in the respiratory tract light therapy unit 102 to support the configured irradiation unit 108 and to adapt the respiratory tract light therapy unit 102 for at will placement of the light transmitting external surface of the configured irradiation unit 108 at a fixed position and desired irradiation direction on the chest 502. Support structure 128 is preferably provided to help secure the respiratory tract light therapy unit 102 to the chest 502 and to make the respiratory tract light therapy unit 102 more comfortable for the subject 500 to wear.

The configured irradiation unit 108 is positioned in the respiratory tract light therapy unit 102 such that it can target specific locations. In the preferred embodiment, the configured irradiation unit 108 is positioned to direct light energy to at least one portion of an in-vivo target selected from the group consisting of a thymus gland, sternal bone marrow and lungs.

As can be seen in FIGS. 1 and 3 to 4 the preferred system of the present invention optionally comprises an intranasal light therapy unit 200 which includes a nose clip 202. The nose clip 202 holds a configured irradiation lens 204 inside one of the nostrils of the subject 500. The configured irradiation lens 204 includes a portable hollow casing having fixed dimensions, a sized internal spatial volume, and an external surface configuration suitable for application to the interior of the nostrils.

The portable casing comprises: (i) a light energy transmitting material which forms at least a portion of the configured external surface for said hollow casing, and (ii) at least one light generating unit entirely housed and contained within said internal spatial volume of said hollow casing and which is capable of generating light energy of at least one preselected wavelength selected from the group consisting of near infrared red light wavelengths and visible red light wavelengths, at a predetermined energy intensity, for a preset time duration, and at a predetermined pulse frequency, collectively on-demand sufficient to penetrate through the nasal tissues and to pass into the blood vessels.

A first connector 300 may be in electrical communication with the configured irradiation unit 108 of the respiratory tract light therapy unit 102. A second connector 400 may be in electrical communication with the configured irradiation lens 204 of the intranasal light therapy unit 200. This allows for on-demand conveyance of direct electrical current from a power source, such as a battery pack 600 or power plug 700 which is plugged into an electrical outlet, through the controller assembly 150 and to the light generating unit(s) in the configured irradiation unit 108, as well as the light generating unit(s) of configured irradiation lens 204 in the intranasal light therapy unit 200.

Experimental Section

A 30 day randomized study was conducted to evaluate the efficacy of a preferred system of the present invention in the treatment of COVID-19 respiratory symptoms.

Subjects for the Study

A total of 280 subjects, aged between 18 and 65 years, participated in the study. All subjects were confirmed to have tested positive for the COVID-19 infection, with moderate to severe symptoms. The subjects are randomized in a 1:1 ratio either to be treated with the invention or receive the standard of care (SOC).

None of the subjects were hospitalized or in need of supplemental oxygen or positive pressure support. Furthermore, none of the subjects were pregnant, had been diagnosed with Chronic Obstructive Pulmonary Disease (COPD), or tested positive for Hepatitis C Virus (HCV), Hepatitis B Virus (HBV) or Human Immunodeficiency Virus (HIV).

Treatment of Subjects

The system of the present invention was administered to subjects for 20 minutes, twice a day for the first 5 days, with each administration was separated by at least 6 hours. Subsequently, subjects were treated once daily for 20 minutes.

An NIR LED module of the preferred system of the present invention was positioned over the manubrium of the sternum to target the upper sternum. An intranasal applicator was positioned inside the left or right nostril of the subject.

Subjects were asked to self-report their symptoms by filling out the Wisconsin Upper Respiratory Symptom Survey (WURSS-44). Oxygen saturation levels at rest were measured.

Results

73 subjects have been randomized into either the treatment group or into the group receiving standard of care. Independent statistical analysis reported that the study is very promising, and is strongly recommended for full completion with the planned 280 subjects.

The scope of the claims should not be limited by the preferred embodiments set forth in the examples, but should be given the broadest interpretation consistent with the description as a whole.

1. A system for improved immunity and treatment of respiratory tract infections in a subject, said system comprising:
a configured irradiation unit comprising a portable hollow casing having fixed dimensions, a sized internal spatial volume and an external surface configuration suitable for application to the chest, said portable hollow casing of the configured irradiation unit being comprised of:
(i) a light energy transmitting material which forms at least a portion of the configured external surface for said hollow casing of the configured irradiation unit; and
(ii) at least one light generating unit housed and contained within said internal spatial volume of said hollow casing of the configured irradiation unit and which is capable of generating light energy of at least one preselected wavelength selected from the group consisting of near infrared light wavelengths and visible red light wavelengths, at a predetermined energy intensity, for a preset time duration, and at a predetermined pulse frequency, collectively on-demand sufficient to penetrate through the skin and to pass to at least one portion of an in-vivo target selected from the group consisting of the thymus gland, sternal bone marrow and lungs,
whereby said configured irradiation unit can emit light energy after application to the chest and achieve passage of said emitted light energy through the skin into the at least one portion of the in-vivo target;
a frame adapted for support of said configured irradiation unit and for at will placement of said light transmitting external surface of said configured irradiation unit at a fixed position and desired irradiation direction on the chest;
a portable controller assembly able to control on-demand delivery of light energy from said configured irradiation unit into at least one portion of the thymus gland, sternal bone marrow and/or lungs in-vivo, said controller assembly including:
(a) a power source of on-demand direct electrical current,
(b) a central processing unit for controlling and directing the flow of such direct electrical current,
(c) at least one connector in electrical communication with the power source for on-demand conveyance of direct electrical current to the central processing unit, and
(d) at least one connector in electrical communication with the configured irradiation units for on-demand conveyance of direct electrical current from said central processing unit to said light generating units.

2. The system of claim 1, said system further comprising:
a configured irradiation lens including:
a portable hollow casing having fixed dimensions, a sized internal spatial volume, and an external surface configuration suitable for in-vivo insertion into the nasal cavity space of a nostril without causing substantial impairment to the subject's ability to breathe and without invading the nasal tissues of the living subject, said portable casing of said configured irradiation lens being comprised of:
(i) a light energy transmitting material which forms at least a portion of the configured external surface for said hollow casing of said configured irradiation lens,
(ii) at least one light generating unit housed and contained within said internal spatial volume of said hollow casing of said configured irradiation lens and which is capable of generating light energy of at least one preselected wavelength selected from the group consisting of near infrared light wavelengths and visible red light wavelengths, at a predetermined energy intensity, for a preset time duration, and at a predetermined pulse frequency, collectively on-demand sufficient to penetrate through the nasal tissues and to pass into the blood vessels,
whereby said configured irradiation lens can emit light energy in any desired direction within the nasal cavity after in-vivo insertion and achieve passage of said emitted light energy from the nasal cavity into at least one portion of the blood vessels in-vivo;
a self-administrable applicator means adapted for support of said configured irradiation lens and for at will placement of said light transmitting external surface of said configured irradiation lens at a fixed position and desired irradiation direction within a nostril adjacent to the internal lining of a subject's nasal cavity;
wherein said portable controller assembly is further able to control on-demand delivery of light energy from said configured irradiation lens.

3. The system of claim 1, wherein the light energy has a wavelength of about 633 nm to 810 nm.

4. The system of claim 1, wherein the respiratory tract infection is COVID-19.

5. A method for improved immunity and treatment of respiratory tract infections in a subject, said method comprising the steps of:
A. obtaining a light energy-emitting apparatus comprised of:
a configured irradiation unit comprising a portable hollow casing having fixed dimensions, a sized internal spatial volume and an external surface configuration suitable for application to the chest, said portable hollow casing of the configured irradiation unit being comprised of:
(i) a light energy transmitting material which forms at least a portion of the configured external surface for said hollow casing of the configured irradiation unit; and
(ii) at least one light generating unit housed and contained within said internal spatial volume of said hollow casing of the configured irradiation unit and which is capable of generating light energy of at least one preselected wavelength selected from the group consisting of near infrared light wavelengths and visible red light wavelengths, at a predetermined energy intensity, for a preset time duration, and at a predetermined pulse frequency, collectively on-demand sufficient to penetrate through the skin and to pass to at least one portion of an in-vivo target selected from the group consisting of the thymus gland, sternal bone marrow and lungs,
whereby said configured irradiation unit can emit light energy after application to the chest and achieve passage of said emitted light energy through the skin into at least one portion of the in-vivo target;
a frame adapted for support of said configured irradiation unit and for at will placement of said light transmitting external surface of said configured irradiation unit at a fixed position and desired irradiation direction on the chest;
a portable controller assembly able to control on-demand delivery of light energy from said configured irradiation unit into at least one portion of the thymus gland and the lungs in-vivo, said controller assembly including:
(a) a power source of on-demand direct electrical current,
(b) a central processing unit for controlling and directing the flow of such direct electrical current,
(c) at least one connector in electrical communication with the power source for on-demand conveyance of direct electrical current to the central processing unit, and
(d) at least one connector in electrical communication with the configured irradiation units for on-demand conveyance of direct electrical current from said central processing unit to said light generating units; and
B. causing said light generating units of said positioned configured irradiation unit to generate light energy of at least one preselected wavelength selected from the group consisting of near infrared light wavelengths and visible red light wavelengths, at a predetermined energy intensity, for a preset time duration, and at a predetermined pulse frequency, collectively on-demand sufficient to penetrate through the subject's skin and to pass into at least one portion of the in-vivo target.

6. The method of claim 5, wherein said light energy-emitting apparatus further comprises:
a configured irradiation lens including:
a portable hollow casing having fixed dimensions, a sized internal spatial volume, and an external surface configuration suitable for in-vivo insertion into the nasal cavity space of a nostril without causing substantial impairment to the subject's ability to breathe and without invading the nasal tissues of the living subject, said portable casing of said configured irradiation lens being comprised of:
(i) a light energy transmitting material which forms at least a portion of the configured external surface for said hollow casing of said configured irradiation lens,
(ii) at least one light generating unit housed and contained within said internal spatial volume of said hollow casing of said configured irradiation lens and which is capable of generating light energy of at least one preselected wavelength selected from the group consisting of near infrared light wavelengths and visible red light wavelengths, at a predetermined energy intensity, and for a preset time duration, and at a predetermined pulse frequency, collectively on-demand sufficient to penetrate through the nasal tissues and to pass into the blood vessels,
whereby said configured irradiation lens can emit light energy in any desired direction within the nasal cavity after in-vivo insertion and achieve passage of said emitted light energy from the nasal cavity into at least one portion of the blood vessels in-vivo;
a self-administrable applicator means adapted for support of said configured irradiation lens and for at will placement of said light transmitting external surface of said configured irradiation lens at a fixed position and desired irradiation direction within a nostril adjacent to the internal lining of a subject's nasal cavity;
wherein said portable controller assembly is further able to control on-demand delivery of light energy from said configured irradiation lens.

7. The method of claim 5, wherein the light energy has a wavelength of about 633 nm to 810 nm.

8. The method of claim 5, wherein the respiratory tract infection is COVID-19.
